(19) United States
(12) Patent Application Publication
(10) Pub. No.: US 2024/0361406 A1
(43) Pub. Date: Oct. 31, 2024

(54) RADIO-FREQUENCY POWER AMPLIFICATION MODULE FOR MAGNETIC RESONANCE SYSTEM AND IMAGING METHOD

(71) Applicant: GE Precision Healthcare LLC, Waukesha, WI (US)

(72) Inventors: Xin Xie, Beijing (CN); Dongliang Yang, Beijing (CN); Yu Liu, Beijing (CN); Yanfang Cai, Beijing (CN); Ning Zhang, Beijing (CN); Chaoya Zhao, Beijing (CN); Alen Wang, Beijing (CN)

(21) Appl. No.: 18/643,885

(22) Filed: Apr. 23, 2024

(30) Foreign Application Priority Data
Apr. 28, 2023 (CN) 202310486294.2

(51) Int. Cl. G01R 33/36; G01R 33/54

(52) U.S. Cl. CPC G01R 33/3614; G01R 33/543

(57) ABSTRACT

A radio-frequency power amplification module for a magnetic resonance system and an imaging method are provided. The radio-frequency power amplification module includes: a power synthesizer, a main amplifier, and an auxiliary amplifier, output ends of the main amplifier and the auxiliary amplifier being both connected to a power synthesis unit; and a controller. The controller is configured to output a control signal according to a required radio-frequency transmission parameter or a scan parameter corresponding to the radio-frequency transmission parameter, so as to adjust a control parameter of the auxiliary amplifier. The radio-frequency power amplification module has high efficiency.

### CROSS REFERENCE TO RELATED APPLICATIONS

The present application claims priority and benefit of Chinese Patent Application No. 202310486294.2 filed on Apr. 28, 2023, which is incorporated herein by reference in its entirety.

### TECHNICAL FIELD

The present invention relates to the field of medical imaging, and in particular to a radio-frequency power amplification module for a magnetic resonance system and an imaging method based on a magnetic resonance system.

### BACKGROUND

Magnetic resonance (MR) technology is one of the primary imaging modalities in modern medicine, having a radio-frequency transmission system for generating a high-power radio-frequency excitation signal, so as to excite nuclei in body tissue of a subject to resonate. Radio-frequency transmission systems typically employ radio-frequency amplifiers to amplify generated low-power signals to high-power radio-frequency signals to achieve radio-frequency excitation.

As shown in FIG. 2, a radio-frequency transmit link 200 for magnetic resonance imaging usually includes a radio-frequency signal generator 210, a radio-frequency power amplifier 220, a T/R switch 230, and a radio-frequency transmit coil 240. The radio-frequency transmit coil may be a body coil or a local coil. The linearity of the radio-frequency transmit link is an important factor that affects the quality of magnetic resonance images. The radio-frequency signal generator 210 in the radio-frequency transmit link is configured to generate a small radio-frequency signal, and the T/R switch 230 is a passive device, therefore, in the radio-frequency transmit link, the radio-frequency power amplifier 220 configured to amplify a signal has the greatest impact on linearity. In other words, the radio-frequency power amplifier needs to have high linearity and maintain fidelity.

Magnetic resonance systems typically employ class B or class AB radio-frequency power amplifiers to compromise between linearity and efficiency. In such a design, the maximum efficiency of the radio-frequency power amplifier will occur at the maximum output power (e.g., the maximum output power while linearity is maintained, or the maximum representative value at this output power, wherein the representative value may be a radio-frequency transmit gain), and the efficiency will drop as radio-frequency transmit power decreases. Therefore, for scan modes that have low radio-frequency transmit power, the efficiency of the radio-frequency power amplifier is also low, meaning higher heat dissipation costs and usage costs.

### SUMMARY

Provided in one aspect of the present invention is a radio-frequency power amplification module for a magnetic resonance system, comprising: a power synthesizer, a main amplifier, and an auxiliary amplifier, output ends of the main amplifier and the auxiliary amplifier being both connected to a power synthesis unit; and a controller. The controller is configured to output a control signal according to a required radio-frequency transmission parameter or a scan parameter corresponding to the radio-frequency transmission parameter, so as to adjust a control parameter of the auxiliary amplifier.

Provided in another aspect of the present invention is an imaging method based on a magnetic resonance system, the magnetic resonance system comprising the radio-frequency power amplification module according to the example above. The method includes adjusting the auxiliary amplifier to the same type as the main amplifier on the basis of a selected transmit coil being a body coil; performing a pre-scan; and determining the radio-frequency transmission parameter or the scan parameter on the basis of the pre-scan. The method further includes outputting the control signal on the basis of the radio-frequency transmission parameter or the scan parameter.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present invention will be better understood by reading the following description of non-limiting examples with reference to the accompanying drawings, wherein

FIG. 1 illustrates a schematic diagram of an exemplary magnetic resonance system 100 according to some examples of the present invention;

FIG. 2 illustrates a block diagram of a radio-frequency transmit link of a magnetic resonance system according to some examples;

FIG. 3 illustrates a trend in which efficiency of a radio-frequency power amplifier changes along with output power;

FIG. 4 illustrates a use frequency distribution of transmit power of a radio-frequency coil;

FIG. 5 illustrates a block diagram of a radio-frequency power amplification module 500 according to some examples of the present invention;

FIG. 6 illustrates a power-efficiency curve chart under different control parameters obtained on the basis of some examples of the present invention;

FIG. 7 illustrates a schematic diagram of a connection between the controller 540 and an auxiliary amplifier 530 in FIG. 1;

FIG. 8 illustrates a schematic structural diagram of a Doherty amplifier according to some examples;

FIG. 9 illustrates an imaging method 900 for a magnetic resonance system according to some examples of the present invention; and

FIG. 10 illustrates an efficiency improvement curve of a radio-frequency power amplifier obtained by using examples of the present invention.

The accompanying drawings illustrate components, systems, and methods described in the MRI method and system. Together with the following description, the accompanying drawings illustrate and explain structural principles, methods and principles described herein. In the accompanying drawings, the thickness and dimensions of the components may be enlarged or otherwise modified for clarity. Well-known structures, materials, or operations are not shown or described in detail to avoid obscuring the components, systems, and methods described.

### DETAILED DESCRIPTION

Specific embodiments of the present invention will be described below. It should be noted that in the specific description of said embodiments, for the sake of brevity and conciseness, the present description cannot describe all of the features of the actual embodiments in detail. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for a person of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless otherwise defined, the technical or scientific terms used in the claims and the description should be as they are usually understood by those possessing ordinary skill in the technical field to which they belong. Terms such as “first”, “second”, and similar terms used in the present description and claims do not denote any order, quantity, or importance, but are only intended to distinguish different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections. Furthermore, it should be understood that references to “an example” or “examples” of the present disclosure are not intended to be construed as excluding the existence of additional implementations that also incorporate the referenced features.

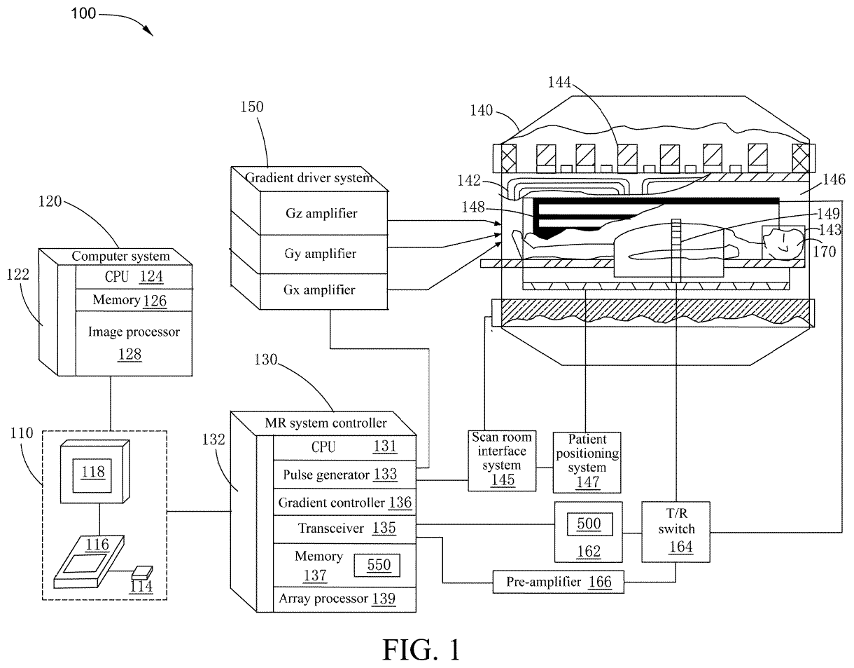

FIG. 1 illustrates a schematic diagram of an exemplary magnetic resonance (MR) system 100 according to some examples of the present invention.

As shown in FIG. 1, an operator workstation 110 is configured to control an operation of the MR system 100, the operator workstation 110 including an input apparatus 114, a control panel 116, and a display 118. The input apparatus 114 may be a joystick, a keyboard, a mouse, a trackball, a touch-activated screen, voice control, or any similar or equivalent input apparatus. The control panel 116 may include a keyboard, a touch-activated screen, voice control, a button, a slider, or any similar or equivalent control apparatus. The operator workstation 110 is coupled to and communicates with a computer system 120, and provides an interface to allow an operator to plan a magnetic resonance scan, display an image, perform image processing, and store data and images. The computer system 120 enables the operator to control (e.g., by means of the input apparatus) the generation and viewing of images and other monitoring information on the display 118, and also to perform human-computer interaction by means of a user interface displayed on the display 118. The human-computer interaction can be used to: input information required for the scan, determine scan parameters, perform operations for image processing, select images, view quality assessment results for the selected images, etc. The above information required for the scan may include information about a scan subject, for example, one or a plurality of gender, age, height, and body weight. The above scan parameters may include one or a plurality of a scan site, a scan sequence, a selected transmit coil, a selected receive coil, a scan sequence, etc.

The computer system 120 includes a plurality of modules that communicate with one another by means of an electrical and/or data connection module 122. The connection module 122 may be a wired communication link, an optical fiber communication link, a wireless communication link, and the like. The computer system 120 may include a central processing unit (CPU) 124, a memory 126, and an image processor 128. In some embodiments, the image processor 128 may be replaced by an image processing function run in the CPU 124. The computer system 120 may be connected to an archive media apparatus, a persistent or backup memory, or a network. The computer system 120 may be coupled to and communicates with a separate MR system controller 130.

The MR system controller 130 includes a set of modules that communicate with one another by means of an electrical and/or data connection module 132. The connection module 132 may be a direct wired communication link, an optical fiber communication link, a wireless communication link, and the like. In an alternative embodiment, modules of the computer system 120 and the MR system controller 130 may be implemented on the same computer system or on a plurality of computer systems. The MR system controller 130 may include a CPU 131, a sequence pulse generator 133 that communicates with the operator workstation 110, a transceiver (or an RF transceiver) 135, a gradient controller 136, a memory 137, and an array processor 139.

The magnetic resonance system 100 may include a resonance assembly 140 and a housing (not shown in the figure) for accommodating the resonance assembly 140. A subject 170 undergoing the magnetic resonance scan may be positioned within a cylindrical imaging volume 146 of the resonance assembly 140. The resonance assembly 140 includes a superconducting magnet having a superconducting coil 144, a radio-frequency coil assembly, and a gradient coil assembly 142. During operation, the superconducting coil 144 provides a static uniform longitudinal magnetic field B₀ throughout the cylindrical imaging volume 146. The radio-frequency coil assembly may include a body coil 148 and a surface coil 149, and may be used to send and/or receive a radio-frequency signal. The radio-frequency coil assembly may further include a local coil 143, for example, a head coil.

The radio-frequency body coil 148 and the local coil 143 may connect to a toggle switch (not shown in the figure), which is controlled by the MR system controller 130 to toggle between the radio-frequency body coil 148 and the local coil 143. In some examples, based on the confirmation of the scan site, the body coil 148 or the local coil 143 may be selected for imaging. For example, when a systemic scan is performed, the body coil 148 may be selected based on an operation on the toggle switch, and when a local scan is performed, the local coil 143 may be selected based on an operation on the toggle switch. The local scan may include, for example, a head scan, an abdominal scan, or a scan of another body part.

The MR system controller 130 may receive a command from the operator workstation 110 to indicate an MR scan sequence that is to be executed during an MR scan. The “scan sequence” above refers to a combination of pulses that have specific intensities, shapes, time sequences, and the like and that are applied when a magnetic resonance imaging scan is executed. The pulses may typically include, for example, a radio-frequency pulse and a gradient pulse. The radio-frequency pulses may include, for example, radio-frequency excitation pulses, radio-frequency refocusing pulses, inverse recovery pulses, etc. These radio-frequency pulses may have different amplitudes, phases, flip angles, etc., based on their different effects. For different magnetic resonance systems, due to differences in hardware design, different radio-frequency transmit power may be needed for the same flip angle requirement. The gradient pulse may include, for example, a gradient pulse used for layer selection, a gradient pulse used for phase encoding, a gradient pulse used for frequency encoding, a gradient pulse used for phase shifting (phase shift), a gradient pulse used for dispersion of phases (dephasing), etc. These gradient pulses may also have different amplitudes and/or durations.

Typically, a plurality of scanning sequences can be pre-set in the magnetic resonance system, so that the sequence suitable for clinical detection requirements can be selected. The clinical detection requirements may include, for example, an imaging site, an imaging function, an imaging effect, and the like. For example, the scan sequence may be determined based on a site to be imaged. For example, when it is determined that the imaging site is a head, a human-computer interaction interface may provide a list of sequences corresponding to the head scan. A suitable sequence may also be further determined from the list based on clinically relevant settings.

The gradient pulse in the scan sequence sent by the pulse generator 133 may be generated by the gradient controller 136 and acts on a gradient driver 150. The gradient driver 150 includes Gₓ, G_{y}, and G_{z} amplifiers, and the like. Each of the Gₓ, G_{y}, and G_{z} gradient amplifiers is used to excite a corresponding gradient coil in the gradient coil assembly 142, so as to generate a magnetic field gradient used to spatially encode an MR signal during an MR scan.

The pulse generator 133 is coupled to and communicates with a scan room interface system 145 that can receive signals from various sensors associated with the state of the resonance assembly 140, and various processors arranged in a scan room. The scan room interface system 145 is further coupled to and communicates with a patient positioning system 147, the patient positioning system 147 sending and receiving a signal to control the movement of a patient table to a desired position to perform the MR scan.

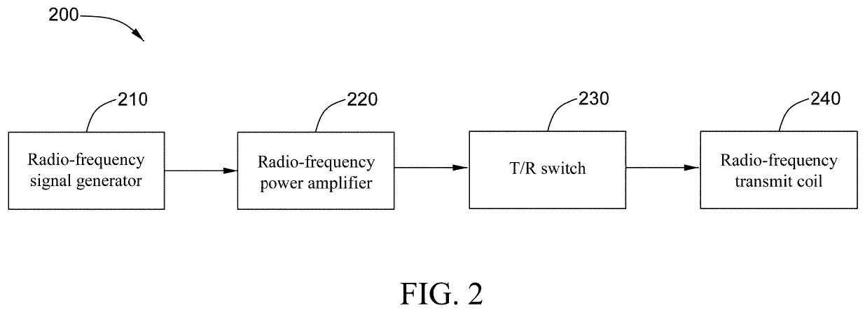

A radio-frequency pulse in the scan sequence sent by the pulse generator 133 may be generated by the transceiver 135 (for example, including the radio-frequency signal generator 210 in FIG. 2), and the radio-frequency pulse is amplified by a radio-frequency power amplifier 162. When the toggle switch is toggled to the body coil 148, the amplified radio-frequency pulse is provided to the body coil 148 via a transmit/receive switch (T/R switch) 164, and the RF body coil 148 then provides a transverse magnetic field B₁. The transverse magnetic field B₁ is substantially perpendicular to B₀ throughout the entire cylindrical imaging volume 146. The transverse magnetic field B₁ is used to excite stimulated nuclei in the body of the scan subject so as to generate an MR signal. When the toggle switch 141 is toggled to the local coil 143, the amplified radio-frequency pulse is provided to the local coil 143, so as to provide the transverse magnetic field B₁ via the local coil 143.

Specifically, the radio-frequency signal generator 210 may generate a corresponding radio-frequency power signal based on a description (e.g., including one or a plurality of an amplitude, a frequency, transmit power, a representative parameter used to represent the transmit power, etc.) of a radio-frequency pulse in a scan sequence that is pre-determined (e.g., selected by a user or determined by performing a pre-scan). The radio-frequency power signal is amplified by the radio-frequency power amplifier 162 to then generate a radio-frequency power amplified signal, which is provided to the RF body coil 148 or the local coil 143 via a radio-frequency power transmission module (e.g., including the T/R switch 164).

As described above, the RF body coil 148, the local coil 143, and the RF surface coil 149 may be used to transmit radio-frequency pulses and/or receive MR signals from the scan subject. The MR signals emitted by excited nuclei in the body of the scan subject may be sensed and received by the RF body coil 148, the local coil 143 or the RF surface coil 149, and then sent back to a preamplifier 166 by means of the T/R switch 164. The T/R switch 164 may be controlled by a signal from the MR system controller 130 to electrically connect, during a transmit mode, the radio-frequency power amplifier 162 to the RF body coil 148 and to connect, during a receive mode, the preamplifier 166 to the RF body coil 148. The T/R switch 164 may further enable the RF surface coil 149 and the local coil 143 to be used in the transmit mode or the receive mode.

In some embodiments, the MR signals sensed and received by the RF body coil 148, the local coil 143, or the RF surface coil 149 and amplified by the preamplifier 166 are demodulated, filtered, and digitized in a receiving portion of the transceiver 135, and transmitted as a raw k-space data array to the memory 137 in the MR system controller 130.

A reconstructed magnetic resonance image may be obtained by transforming/processing the stored raw k-space data. For each image to be reconstructed, the data is rearranged into separate k-space data arrays, and each of said separate k-space data arrays is input to the array processor 139, the array processor being operated to transform the data into an array of image data by Fourier transform.

The array processor 139 uses transform methods, most commonly Fourier transform, to create images from the received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and MR system controller 130 may be implemented on the same computer system or on a plurality of computer systems. It should be understood that the MR system 100 shown in FIG. 1 is intended for illustration. Suitable MR systems may include more, fewer, and/or different components.

The MR system controller 130 and the image processor 128 may separately or collectively include a computer processor and a storage medium. The storage medium records a program that is for predetermined data processing and that is to be executed by the computer processor. For example, the storage medium may store thereon a program used to implement scanning processing (such as a scan flow, an imaging sequence, parameter determination, etc.), image reconstruction, image processing, and the like. The described storage medium may include, for example, a ROM, a floppy disk, a hard disk, an optical disk, a magneto-optical disk, a CD-ROM, or a non-volatile memory card.

The MR system controller 130 controls the various components of the magnetic resonance system 100 to implement corresponding operations, so as to carry out the scan process for the scan subject. This scan process may include a pre-scan and a formal scan after the pre-scan.

One or a plurality of scan sequences may be performed during the pre-scan and the formal scan, wherein the pre-scan may include a scout scan or a calibration scan performed before the scout scan. During the scout scan, at least one of a coronal scout image, a sagittal scout image, and an axial scout image of the subject may be acquired, and based on this scout image, scan parameters of the formal scan, such as the scan range, a scan sequence, the radio-frequency power/a radio-frequency transmit gain, etc., of the formal scan, may be determined. During the calibration scan, frequency adjustment may be performed to determine the Larmor frequency of proton resonance of the current scan, the subsequent scout scan, and the formal scan on the basis of magnetic resonance signal feedback at different frequencies, and radio-frequency transmit intensity adjustment may be performed to determine the radio-frequency transmit power/a radio-frequency transmit gain of the current scan on the basis of magnetic resonance signal feedback at different radio-frequency transmit intensities. The transmit gain above may be used to represent the radio-frequency transmit power. For example, different transmit power is represented by different transmit gains. Different magnetic resonance systems may use different parameters to represent the same transmit power, and the values of the representative parameters of different magnetic resonance systems may be different. The different magnetic resonance systems above refer to magnetic resonance systems having, for example, different brands, production manufacturers, product models, etc.

As a non-limiting example, a transmitting portion in the transceiver 135, the radio-frequency power amplifier 162, the toggle switch, the T/R switch 164, and the like shown in FIG. 1 constitute at least a portion of a radio-frequency transmit link, which may be as shown in FIG. 2. In some embodiments, the radio-frequency transmit link may further include a decoupled switch (not shown in the figure) to provide decoupling or signal isolation between the RF body coil 148, the local coil 143, or the RF surface coil 149 and a receive loop during radio-frequency transmission. Furthermore, the receiving portion in the transceiver 135, the preamplifier 166, the T/R switch 164, and the like shown in FIG. 1 constitute at least a portion of a radio-frequency receive link. One or a plurality of modules/elements/assemblies of the radio-frequency transmit link and radio-frequency receive link are integrated in a resonance assembly, for example, disposed in a magnet housing of a scan room.

As described above, in magnetic resonance imaging, the radio-frequency power amplifier 162 needs to have high linearity to maintain fidelity, and also needs to meet a certain efficiency requirement. Because the power of the radio-frequency amplifier is particularly high, for example, 10 KW, if the efficiency is low, this means that a great deal of power is wasted due to heating, which causes not only high requirements for a power supply, but also high requirements for heat dissipation or cooling. The efficiency of the radio-frequency amplifier is low for several reasons as follows.

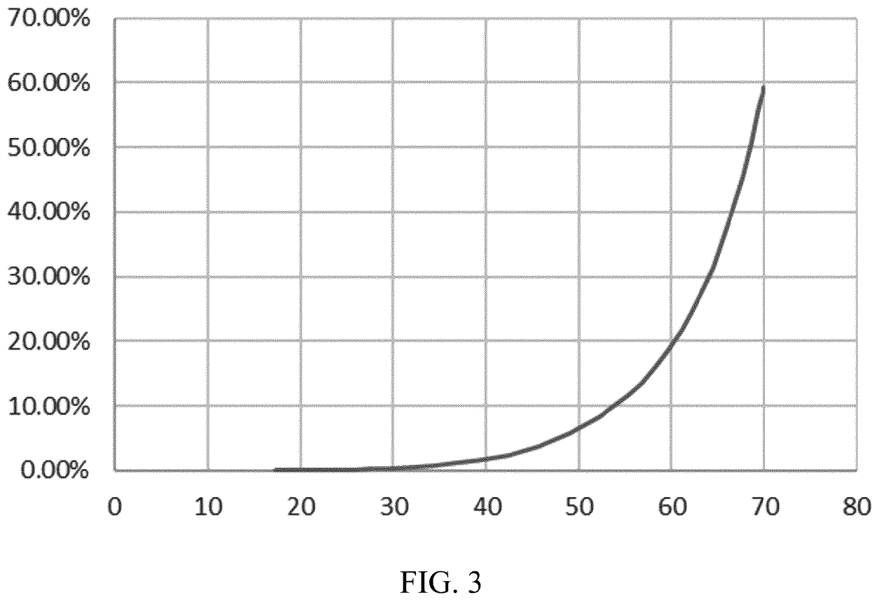

In one aspect, FIG. 3 illustrates a trend in which efficiency of a radio-frequency power amplifier changes along with changes in output power. The horizontal coordinates are the output power (in the unit of dBm), and the vertical coordinates are the efficiency (in the unit of %). As illustrated, as the output power increases, the efficiency also gradually increases. However, the nominal power (or full power) of a body coil is, for example, 10 KW (for example, 70 dBm), and the nominal power of a local coil is, for example, 2 KW (for example, 63 dBm). It can be seen from FIG. 3 that the efficiency corresponding to 70 dBm is about 60%, and the efficiency corresponding to 63 dBm is only about 24%. In other words, even if both the body coil and the local coil operate at the nominal power, the efficiency of supply to a radio-frequency amplifier of the local coil is too low, which means that too much power is wasted by means of heating.

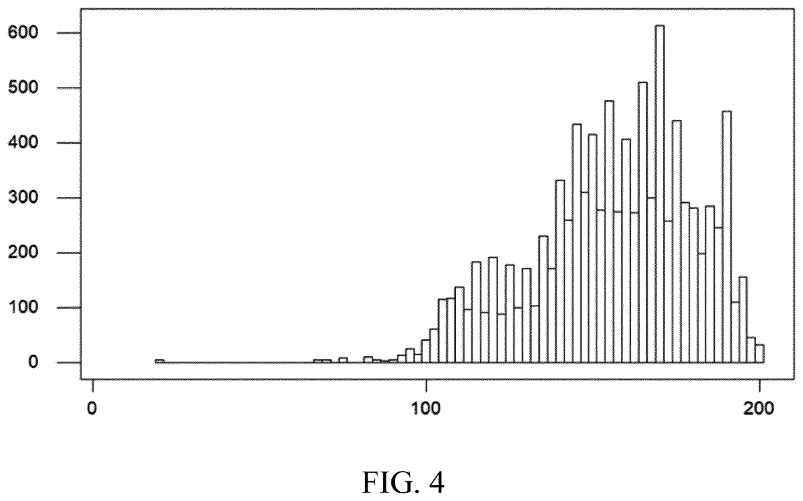

In another aspect, FIG. 4 illustrates a use frequency distribution of transmit power of a radio-frequency coil. The horizontal coordinates are transmit gains (TGs). The TG is an amount for controlling the magnitude of the transmit power, and when TG=200, it means that the power at such time is the nominal power. The vertical coordinates are the use frequencies. As illustrated, the transmit power of the radio-frequency coil is not always the nominal power. In other words, the output power of the radio-frequency amplifier is not always the nominal power. It can be seen from FIG. 4 that the range of the TG is mostly between 100 and 200. Therefore, the efficiency of the radio-frequency power amplifier that does not operate at the nominal power is relatively lower.

There are generally class A, class B, class AB, class C, etc., radio-frequency power amplifiers. A class A amplifiers have good linearity but poor efficiency. Class B amplifiers have poorer linearity but better efficiency than the class A amplifiers. Class AB amplifiers balances linearity and efficiency. Class C amplifiers have poor linearity. Although a class B or AB amplifier is commonly selected for use in magnetic resonance imaging to balance linearity and efficiency, however, according to the analysis above, the radio-frequency power amplifier still has low average efficiency due to the complex system design and working principles of magnetic resonance.

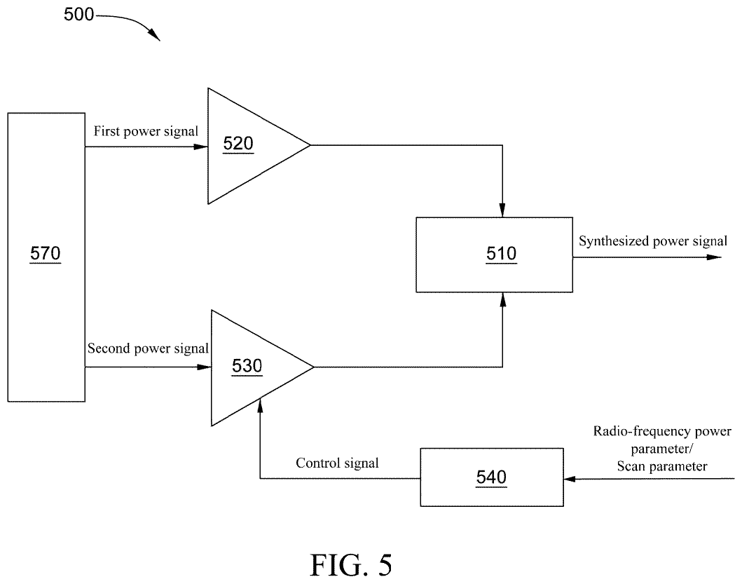

FIG. 5 illustrates a radio-frequency power amplification module 500 according to some examples of the present invention. The radio-frequency power amplification module 500 may be applied to the radio-frequency power amplifier shown in FIG. 1 or FIG. 2 to improve the efficiency of the radio-frequency power amplifier while ensuring the linearity of the radio-frequency power amplifier. Specifically, the radio-frequency power amplification module 500 in the examples of the present invention may be used as at least one unit constituting the topology of an amplifier. For example, the topology of an amplifier may be formed by using a plurality of modules 500.

With reference to FIG. 5, the radio-frequency power amplification module 500 includes a power synthesizer 510, a main amplifier 520, an auxiliary amplifier 530, and a controller 540. Output ends of the main amplifier 520 and the auxiliary amplifier 530 are both connected to a power synthesis unit 510. The controller 540 is connected to the auxiliary amplifier 530. The controller 540 is configured to output a control signal to the auxiliary amplifier 530 based on a required radio-frequency transmission parameter or a scan parameter corresponding to the radio-frequency transmission parameter, so as to adjust a control parameter of the auxiliary amplifier 530.

The required radio-frequency transmission parameter above may be determined based on a specific system or clinical need, which may differ due to different system configurations and scan flows/parameter settings.

In the solution above, by means of providing the main amplifier and the auxiliary amplifier that are connected to the same power synthesizer, and by means of adjusting the control parameter of the auxiliary amplifier based on the required radio-frequency transmission parameter or the scan parameter related to the radio-frequency transmission parameter, the auxiliary amplifier can be dynamically adjusted, avoiding the problem of low linearity or low efficiency caused by an amplifier always operating under a fixed parameter/mode/state when the radio-frequency transmission parameter or the corresponding scan parameter changes.

In an example, the main amplifier 520 and the auxiliary amplifier 530 are respectively configured to receive a first power signal to be amplified and a second power signal to be amplified. The power synthesis unit 510 is configured to perform power synthesis on the power signals processed (e.g., amplified) via the main amplifier 520 and the auxiliary amplifier 530, to generate a synthesized power signal.

As an example, the main amplifier 520 and the auxiliary amplifier 530 may respectively receive the first power signal and the second power signal from a signal output unit 570, and the signal output unit 570 may be, for example, a power distributor. Specifically, the power distributor is configured to divide a radio-frequency power signal received (e.g., from an upper-stage amplifier array or from a radio-frequency signal generator) into two radio-frequency component signals according to a certain power distribution ratio and phase relationship, and respectively transmit the two radio-frequency component signals to the main amplifier 520 and the auxiliary amplifier 530. The main amplifier 520 and the auxiliary amplifier 530 amplify the received radio-frequency component signals, respectively, to synthesize the two amplified radio-frequency component signals via the power synthesizer 510, and output a synthesized radio-frequency power signal. In addition, by means of the synthesizer, the auxiliary amplifier 530 performs active load pulling on the main amplifier 520. The synthesized signal may further serve as an input signal of a next-stage amplifier, or may be directly transmitted to a radio-frequency transmit coil for radio-frequency excitation.

In other examples, the first power signal and the second power signal may also be provided via a separate signal output unit.

In an example, the radio-frequency transmission parameter may include required radio-frequency transmit power or a representative parameter used to represent the radio-frequency transmit power. One example of the representative parameter may include a radio-frequency transmit gain, i.e., the TG above. Usually, the value of the representative parameter is set between a maximum value and a minimum value, wherein the maximum value and the minimum value respectively represent the maximum rated power and the minimum transmit power of the magnetic resonance radio-frequency transmission system. In other examples, the radio-frequency transmit power may also be represented in other manners, which may differ due to different manufacturer definitions.

In an example, the required radio-frequency transmission parameter may be determined by performing the above pre-scan on a scan subject via the magnetic resonance system. The controller 540 may output different control signals on the basis of different radio-frequency transmission parameters.

In another example, the scan parameter may include the type of a selected radio-frequency transmit coil. For example, the controller 540 may set the control signal to have a first value on the basis of a selected body coil, and set the control signal to have a second value on the basis of a selected local coil. The scan parameter may also include a scan site. For example, the controller 540 may set the control signal to have a first value on the basis of a systemic scan mode and set the control signal to a have second value on the basis of a local scan mode. In addition, furthermore, the controller 540 may change the second value accordingly based on different local coils or different local scan sites (e.g., having different radio-frequency transmit powers).

In another example, the scan parameter may include information about a scan subject, for example, the height, body weight, gender, age, etc., of the subject described above. The controller 540 may output a corresponding control signal according to the radio-frequency transmit power determined based on the information. For example, when a scan is performed on a big scan subject and a small scan subject, high or low radio-frequency transmit power may correspondingly exist. Based on the acquired information, the control outputs a control signal corresponding to the high radio-frequency transmit power or outputs a control signal corresponding to the low radio-frequency transmit power.

The controller 540 may be integrated with the computer system 120 or the system controller 130, for example, as a portion of the computer system 120 or the system controller 130. The controller 540 is configured to be capable of communicating with at least a portion of the computer system 120 or the system controller 130, so as to be capable of receiving a scan parameter set by an operator or automatically determined by the system, and transmitting the control signal to the radio-frequency transmit link.

In an example, the controller 540 may output a control signal on the basis of a predetermined correspondence 550, wherein the correspondence 550 includes the correspondences between different radio-frequency transmission parameters and different control signals. The correspondence 550 may further include the correspondence between a scan parameter and a control signal. As the scan parameter corresponds to a radio-frequency transmission parameter, the correspondence between the scan parameter and the control signal may also be interpreted as the correspondence between the radio-frequency transmission parameter and the control signal.

Furthermore, the correspondences may be stored in the form of a lookup table or curve chart. As shown in FIG. 1, the correspondences 550 may be stored in the memory 137 or the memory 126 in the form of a lookup table or a curve chart.

For example, the correspondences may be obtained by acquiring the maximum linear power under different control signals, and the controller 540 may determine corresponding maximum linear power on the basis of the required radio-frequency transmission parameter or the scan parameter, and determine a corresponding control signal on the basis of the determined maximum linear power. The maximum linear power above refers to the maximum available power while linearity is maintained. The maximum linear power may be further determined by, for example, measuring peak power or compression point power of the auxiliary amplifier 530 under different control parameters. The compression point above may be set as required. For example, the compression point may be set to P1 dB or P2 dB (dropping a gain to 1 dB or 2 dB lower than a linear gain), depending on a linear compensation capability of the system. In addition, output power at the compression point is tested, and is used as the maximum linear power above.

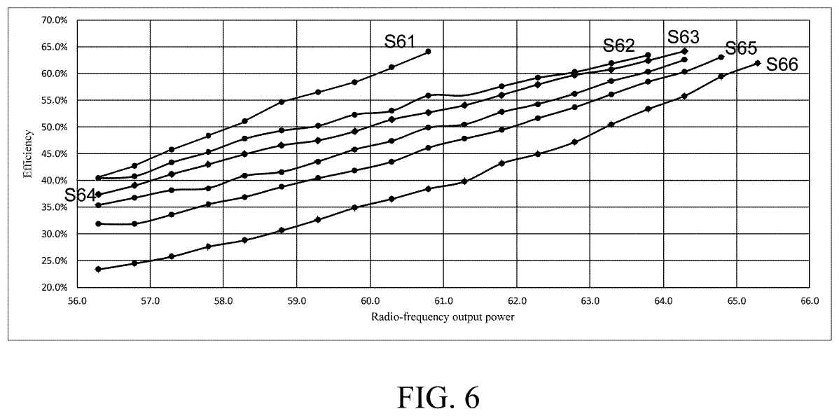

FIG. 6 illustrates a power-efficiency curve chart under different control parameters obtained based on some examples of the present invention. The horizontal coordinates are the radio-frequency output power of the auxiliary amplifier, and the vertical coordinates are efficiency. S61 to S66 are respectively power-efficiency curves under six different control parameters. The value of the horizontal coordinate corresponding to the highest point in each of the curves S61 to S66 is the maximum linear power under a corresponding control parameter, while the value of the vertical coordinate corresponding to each highest point is the maximum efficiency under the corresponding control parameter.

Based on acquiring such a curve or discrete values forming the curve, the correspondence between a control signal (corresponding to a control parameter of the auxiliary amplifier) and a required radio-frequency transmission parameter or a scan parameter (including at least one maximum linear power corresponding to the required radio-frequency transmission parameter or the scan) can be obtained. During actual application, the corresponding radio-frequency transmission parameter may be determined based on the scan parameter, and the corresponding control signal (e.g., corresponding to a control parameter corresponding to any curve in FIG. 6) may be searched for based on the determined radio-frequency transmission parameter, and the corresponding control signal may be searched for directly based on the scan parameter.

In this way, the auxiliary amplifier can be dynamically adjusted based on the radio-frequency transmission parameter or the scan parameter, so that the auxiliary amplifier can operate in a linear zone and have the maximum working efficiency under different transmit power requirements, and the radio-frequency power amplification module also has good linearity and high working efficiency.

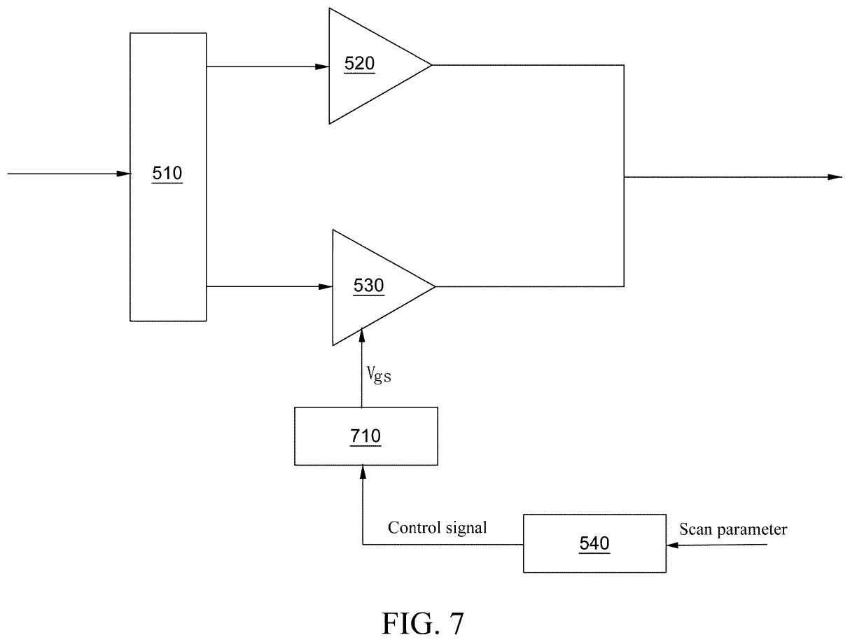

In an example, the control parameter includes a gate-source voltage of the auxiliary amplifier 530. FIG. 7 illustrates a schematic diagram of a connection between the controller 540 and the auxiliary amplifier 530, wherein a power supply unit 710 is provided. One end of the power supply unit 710 is connected to the controller 540 to receive the control signal, and the other end is connected to the gate of the auxiliary amplifier 530 to provide the auxiliary amplifier 530 with a gate-source voltage (a voltage between the gate and the source) V_{gs} as the control parameter. The control signal is used to adjust the value of the gate-source voltage V_{gs} to further adjust a quiescent current of the auxiliary amplifier 530, ensuring that under a current scan parameter, the auxiliary amplifier has the maximum working efficiency within a linear range. The power supply unit 710 may be, for example, an analog-to-digital converter.

In another example, the quiescent current may be directly used as the control parameter of the auxiliary amplifier 530, i.e., the control signal may be used to directly adjust the quiescent current of the auxiliary amplifier.

As described above, a lookup table may be configured based on the correspondences above. However, the following intermediate data associated with the correspondences may also be recorded in the lookup table as required, for example: an electrical characteristic value (e.g., a gate-source voltage, a quiescent current, etc.) related to a control parameter, a control parameter, a radio-frequency transmit gain, radio-frequency transmit power associated with the radio-frequency transmit gain, a scan site, information about a scan subject, a selected transmit coil, etc. During actual operations, a corresponding control signal may also be determined based on any intermediate data as an index.

The main amplifier 520 may be a class B or class AB amplifier having high efficiency and linearity. In an example, the type of the auxiliary amplifier 530 may be adjusted based on the scan parameter, to further adjust the efficiency thereof.

For example, when the radio-frequency transmit power corresponding to the scan parameter or the radio-frequency transmission parameter is greater than a high value, the controller 540 adjusts the auxiliary amplifier to the same type as the main amplifier 520, for example, a class B or class AB amplifier. The high value is greater than (P−1) dB, where P is the rated maximum power, or a power value corresponding to the maximum representative parameter. Specifically, the quiescent current of the auxiliary amplifier 530 may be made equal to a quiescent current of the main amplifier 520 by means of the control signal.

When the radio-frequency transmit power corresponding to the radio-frequency transmission parameter or the scan parameter is less than a high value and greater than a low value, the controller 540 adjusts the auxiliary amplifier 530 to a class C adjustable amplifier. The high value is greater than (P−1) dB, and the low value is less than (P−6) dB. Based on different scan parameters, a control parameter of the class C adjustable amplifier 530 may be further adjusted to satisfy linearity and the maximum working efficiency. Specifically, the controller is configured to output a control signal according to the required radio-frequency transmission parameter or the scan parameter corresponding to the radio-frequency transmission parameter, so as to adjust the control parameter of the class C adjustable amplifier. The controller 540 may change or adjust, based on the required radio-frequency transmission parameter or the scan parameter (e.g., different local scan sites, local coils, different configured transmit power/different transmit gains, etc.) corresponding to the radio-frequency transmission parameter, the control signal that is output to the class C amplifier, for example, may adjust the control signal based on the lookup table above.

When the radio-frequency transmit power corresponding to the radio-frequency transmission parameter or the scan parameter is less than a low value, the controller 540 turns off the auxiliary amplifier 530, and specifically, may adjust the quiescent current of the auxiliary amplifier 530 to 0. The low value is less than (P−6) dB.

In an example, the main amplifier 520 may be a carrier power amplifier, and the auxiliary amplifier 530 may be a peak power amplifier.

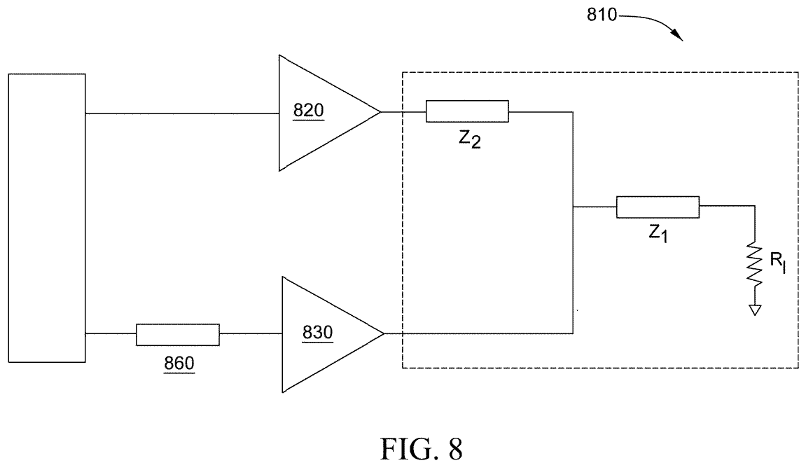

In an example, the power synthesizer 510, the main amplifier 520, and the auxiliary amplifier 530 may be connected to become a Doherty amplifier. The structure of the Doherty amplifier may be shown in FIG. 8, which includes a power synthesizer 810, a main amplifier 820, and an auxiliary amplifier 830. The power synthesizer 810 includes an impedance converter Z₂, a matching network Z₁, and a load resistor Rt. An input end of the auxiliary amplifier 830 is further connected to a phase shifter 860. The impedance converter Z₂ and the phase shifter 860 above may both be quarter-wavelength lines. Specifically, the means of connection and structure of the synthesizer 510, the main amplifier 520, and the auxiliary amplifier 530 may be similar to the corresponding means of connection and structure shown in FIG. 8. Those skilled in the art should understand that the Doherty amplifier has higher efficiency than a single conventional power amplifier, but may cause unacceptable linearity when applied to a magnetic resonance system. Moreover, because the Doherty amplifier has a fixed amplifier type and fixed working parameters, application of the Doherty amplifier to magnetic resonance imaging is limited.

According to the description above, an example of the present invention may further provide the controller 540, and the controller 540 is set to adjust the control parameter of the auxiliary amplifier on the basis of the radio-frequency transmission parameter or the corresponding scan parameter, thereby having high working efficiency while ensuring the linearity of the auxiliary amplifier. The examples of the present invention overcome the limitations above, so that the Doherty amplifier is applied to the magnetic resonance system to achieve amplifier efficiency and linearity that both satisfy requirements.

According to the description above, an example of the present invention may further provide a magnetic resonance system, which includes a radio-frequency power amplifier. The radio-frequency power amplifier may include the amplifier 162 shown in FIG. 1. The radio-frequency power amplifier 162 includes one or more radio-frequency power amplification modules 500.

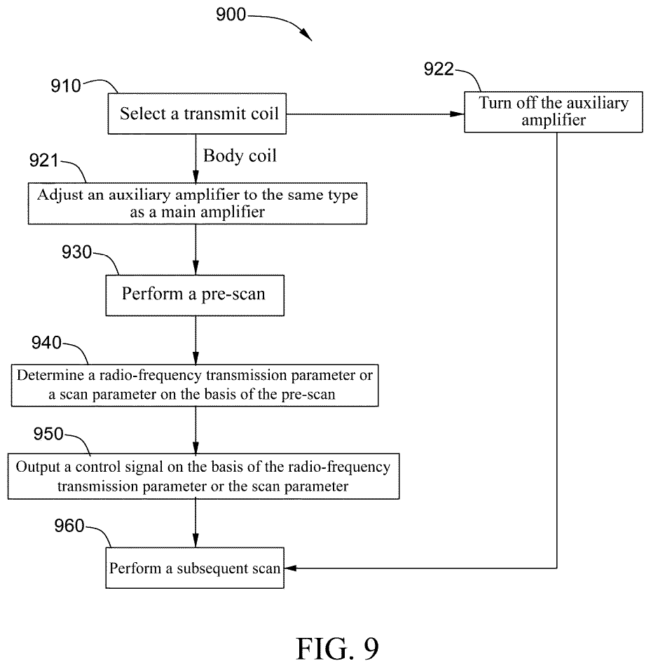

FIG. 9 illustrates an imaging method 900 of a magnetic resonance system according to some examples of the present invention. The magnetic resonance system may include any one of the examples of the magnetic resonance system 100 above. At step 910, a transmit coil is selected, which may be determined by scan parameter settings performed by a user by means of an operator workstation, or determined by automatic selection of the transmit coil in any manner. For example, selection may be performed between a body coil or multiple different local coils.

At step 921, the auxiliary amplifier 530 is adjusted to the same type as the main amplifier 520 based on the selected transmit coil being a body coil. For example, the auxiliary amplifier may be a class B or class AB amplifier. Specifically, the auxiliary amplifier 530 may be adjusted to have the same static operating point as the main amplifier 520.

At step 922, the auxiliary amplifier is turned off based on the selected transmit coil being a local coil.

At step 930, a pre-scan is performed. As described above, the pre-scan may include at least one of a calibration scan and a scout scan.

At step 940, the radio-frequency transmission parameter or the scan parameter is determined based on the pre-scan. For example, radio-frequency transmit power can be determined via the pre-scan, or corresponding radio-frequency transmit power and another parameter, such as a transmit gain, corresponding to the radio-frequency transmit power can be determined based on a scan range, a scan sequence, etc., that are determined in the pre-scan.

At step 950, the control signal is output based on the radio-frequency transmission parameter or the scan parameter. For example, the control signal may be determined by means of the lookup table or curve chart described above. Specifically, in step 950: when the radio-frequency transmit power corresponding to the radio-frequency transmission parameter or the scan parameter is greater than the high value, the controller 540 adjusts the auxiliary amplifier to have the same operating point as the main amplifier, for example, adjusts the auxiliary amplifier to a class B or class AB amplifier; when the radio-frequency transmit power corresponding to the radio-frequency transmission parameter or the scan parameter is less than the high value and greater than the low value, the controller 540 adjusts the auxiliary amplifier 530 to a class C adjustable amplifier, wherein a control parameter of the class C adjustable amplifier may further change on the basis of a change in the scan parameter; and when the radio-frequency transmit power corresponding to the radio-frequency transmission parameter or the scan parameter is less than the low value, the controller 540 turns off the auxiliary amplifier 530.

After step 950, a subsequent scan may further be performed, and any of the above-described settings of the auxiliary amplifier may be employed in the subsequent scan to ensure the imaging quality of the subsequent scan. This subsequent scan may include a scout scan or formal scan after a calibration scan.

By means of this method 900, a control parameter of the radio-frequency power amplification module can be dynamically adjusted based on scan parameters determined at different scan phases, thereby improving efficiency while ensuring linearity.

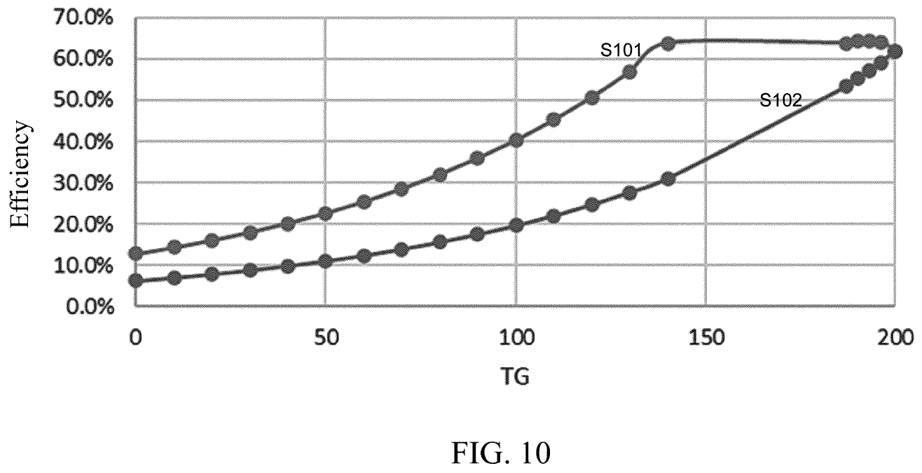

FIG. 10 illustrates an efficiency improvement curve S101 of a radio-frequency power amplifier obtained by using examples of the present invention. The horizontal coordinates represent transmit gains TG corresponding to (or used to represent radio-frequency transmit power) radio-frequency transmit power, and the vertical coordinates represent efficiency. Compared with a conventional radio-frequency power amplifier efficiency curve S102, in the radio-frequency power amplification module of the present invention, the efficiency is improved at each transmit gain (especially when the transmit gain is small).

In addition to any previously indicated modifications, many other variations and replacement arrangements may be devised by those skilled in the art without departing from the substance and scope of the present description, and the appended claims are intended to encompass such modifications and arrangements. Therefore, although the information has been described above in specifics and details in connection with what is currently considered to be the most practical and preferred aspects, it will be apparent to those of ordinary skill in the art that many modifications can be made, including, but not limited to, modifications to the form, function, mode of operation, and use, without departing from the principles and concepts set forth herein. Likewise, as used herein, in all respects, the examples and embodiments are intended to be illustrative only and should not be construed as limiting in any way.

The purpose of providing the above specific examples is to facilitate understanding of the content disclosed in the present invention more thoroughly and comprehensively, but the present invention is not limited to these specific examples. Those skilled in the art should understand that various modifications, equivalent replacements, and changes can also be made to the present invention and should be included in the scope of protection of the present invention as long as these changes do not depart from the spirit of the present invention.

1. A radio-frequency power amplification module for a magnetic resonance system, comprising:
a power synthesizer, a main amplifier, and an auxiliary amplifier, output ends of the main amplifier and the auxiliary amplifier being both connected to a power synthesis unit; and
a controller, the controller being configured to output a control signal according to a required radio-frequency transmission parameter or a scan parameter corresponding to the radio-frequency transmission parameter, so as to adjust a control parameter of the auxiliary amplifier.

2. The radio-frequency power amplification module according to claim 1, wherein the radio-frequency transmission parameter comprises radio-frequency transmit power or a representative parameter used to represent the radio-frequency transmit power.

3. The radio-frequency power amplification module according to claim 2, the representative parameter comprising a radio-frequency transmit gain.

4. The radio-frequency power amplification module according to claim 1, wherein the radio-frequency transmission parameter is determined by pre-scanning a scan subject via the magnetic resonance system.

5. The radio-frequency power amplification module according to claim 1, wherein the scan parameter comprises the type of a selected transmit coil or a scan site.

6. The radio-frequency power amplification module according to claim 1, wherein the scan parameter comprises information about a scan subject.

7. The radio-frequency power amplification module according to claim 1, wherein the controller is configured to output the control signal on the basis of a predetermined correspondence, and the correspondence comprises correspondences between different radio-frequency transmission parameters and different control signals.

8. The radio-frequency power amplification module according to claim 7, wherein
the correspondences are obtained by acquiring the maximum linear power under different control signals, and the controller determines corresponding maximum linear power on the basis of the required radio-frequency transmission parameter or the scan parameter, and determines a corresponding control signal on the basis of the determined maximum linear power.

9. The radio-frequency power amplification module according to claim 7, wherein the correspondences are stored as a lookup table or a curve chart.

10. The radio-frequency power amplification module according to claim 1, wherein the control parameter comprises a gate-source voltage of the auxiliary amplifier.

11. The radio-frequency power amplification module according to claim 10, wherein the auxiliary amplifier receives the gate-source voltage via a power supply unit, and the controller is connected to the power supply unit and provides the control signal to the power supply unit to control the gate-source voltage provided by the power supply unit.

12. The radio-frequency power amplification module according to claim 1, wherein the control parameter comprises a quiescent current of the auxiliary amplifier.

13. The radio-frequency power amplification module according to claim 1, wherein when radio-frequency transmit power corresponding to the radio-frequency transmission parameter or the scan parameter is greater than a high value, the controller adjusts the auxiliary amplifier to the same type as the main amplifier, the high value being greater than (P−1) dB, wherein P is the rated maximum power.

14. The radio-frequency power amplification module according to claim 13, wherein the quiescent current of the auxiliary amplifier is equal to a quiescent current of the main amplifier.

15. The radio-frequency power amplification module according to claim 1, wherein when radio-frequency transmit power corresponding to the radio-frequency transmission parameter or the scan parameter is less than a high value and greater than a low value, the controller adjusts the auxiliary amplifier to a class C adjustable amplifier, the high value being greater than (P−1) dB, and the low value being less than (P−6) dB, wherein P is the rated maximum power.

16. The radio-frequency power amplification module according to claim 15, wherein
the controller is configured to output a control signal according to the required radio-frequency transmission parameter or the scan parameter corresponding to the radio-frequency transmission parameter, so as to adjust a control parameter of the class C adjustable amplifier.

17. The radio-frequency power amplification module according to claim 1, wherein when radio-frequency transmit power corresponding to the radio-frequency transmission parameter or the scan parameter is less than a low value, the controller turns off the auxiliary amplifier, the low value being less than (P−6) dB, wherein P is the rated maximum power.

18. The radio-frequency power amplification module according to claim 1, wherein the power synthesizer, the main amplifier, and the auxiliary amplifier are connected to become a Doherty amplifier.

19. An imaging method based on a magnetic resonance system, the magnetic resonance system comprising the radio-frequency power amplification module according to claim 1, and the method comprising:
adjusting the auxiliary amplifier to the same type as the main amplifier on the basis of a selected transmit coil being a body coil;
performing a pre-scan;
determining the radio-frequency transmission parameter or the scan parameter on the basis of the pre-scan; and
outputting the control signal on the basis of the radio-frequency transmission parameter or the scan parameter.

20. The method according to claim 19, further comprising:
turning off the auxiliary amplifier on the basis of the selected transmit coil being a local coil.
